SEQUENCE LISTING (2 sequences)

SEQ ID NO: 1 - DNA, 3117 bp, Homo sapiens
atgacttcctcgctgcagcggccctggcgggtgccctggctaccatggaccatcctgctggtcagcactgcggctgcttcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagacaataatcattgctttggcatcagtctctgtattagctgttttgatagttgccttatgctttggatacagaatgttgacaggagaccgtaaacaaggtcttcacagtatgaacatgatggaggcagcagcatccgaaccctctcttgatctagataatctgaaactgttggagctgattggccgaggtcgatatggagcagtatataaaggctccttggatgagcgtccagttgctgtaaaagtgttttcctttgcaaaccgtcagaattttatcaacgaaaagaacatttacagagtgcctttgatggaacatgacaacattgcccgctttatagttggagatgagagagtcactgcagatggacgcatggaatatttgcttgtgatggagtactatcccaatggatctttatgcaagtatttaagtctccacacaagtgactgggtaagctcttgccgtcttgctcattctgttactagaggactggcttatcttcacacagaattaccacgaggagatcattataaacctgcaatttcccatcgagatttaaacagcagaaatgtcctagtgaaaaatgatggaacctgtgttattagtgactttggactgtccatgaggctgactggaaatagactggtgcgcccaggggaggaagataatgcagccataagcgaggttggcactatcagatatatggcaccagaagtgctagaaggagctgtgaacttgagggactgtgaatcagctttgaaacaagtagacatgtatgctcttggactaatctattgggagatatttatgagatgtacagacctcttcccaggggaatccgtaccagagtaccagatggcttttcagacagaggttggaaaccatcccacttttgaggatatgcaggttctcgtgtctagggaaaaacagagacccaagttcccagaagcctggaaagaaaatagcctggcagtgaggtcactcaaggagacaatcgaagactgttgggaccaggatgcagaggctcggcttactgcacagtgtgctgaggaaaggatggctgaacttatgatgatttgggaaagaaacaaatctgtgagcccaacagtcaatccaatgtctactgctatgcagaatgaacgcaacctgtcacataataggcgtgtgccaaaaattggtccttatccagattattcttcctcctcatacattgaagactctatccatcatactgacagcatcgtgaagaatatttcctctgagcattctatgtccagcacacctttgactataggggaaaaaaaccgaaattcaattaactatgaacgacagcaagcacaagctcgaatccccagccctgaaacaagtgtcaccagcctctccaccaacacaacaaccacaaacaccacaggactcacgccaagtactggcatgactactatatctgagatgccatacccagatgaaacaaatctgcataccacaaatgttgcacagtcaattgggccaacccctgtctgcttacagctgacagaagaagacttggaaaccaacaagctagacccaaaagaagttgataagaacctcaaggaaagctctgatgagaatctcatggagcactctcttaaacagttcagtggcccagacccactgagcagtactagttctagcttgctttacccactcataaaacttgcagtagaagcaactggacagcaggacttcacacagactgcaaatggccaagcatgtttgattcctgatgttctgcctactcagatctatcctctccccaagcagcagaaccttcccaagagacctactagtttgcctttgaacaccaaaaattcaacaaaagagccccggctaaaatttggcagcaagcacaaatcaaacttgaaacaagtcgaaactggagttgccaagatgaatacaatcaatgcagcagaacctcatgtggtgacagtcaccatgaatggtgtggcaggtagaaaccacagtgttaactcccatgctgccacaacccaatatgccaataggacagtactatctggccaaacaaccaacatagtgacacatagggcccaagaaatgttgcagaatcagtttattggtgaggacacccggctgaatattaattccagtcctgatgagcatgagcctttactgagacgagagcaacaagctggccatgatgaaggtgttctggatcgtcttgtggacaggagggaacggccactagaaggtggccgaactaattccaataacaacaacagcaatccatgttcagaacaagatgttcttgcacagggtgttccaagcacagcagcagatcctgggccatcaaagcccagaagagcacagaggcctaattctctggatctttcagccacaaatgtcctggatggcagcagtatacagataggtgagtcaacacaagatggcaaatcaggatcaggtgaaaagatcaagaaacgtgtgaaaactccctattctcttaagcggtggcgcccctccacctgggtcatctccactgaatcgctggactgtgaagtcaacaataatggcagtaacagggcagttcattccaaatccagcactgctgtttaccttgcagaaggaggcactgctacaaccatggtgtctaaagatataggaatgaactgtctgtga

SEQ ID NO: 2 - protein, 1038 aa, Homo sapiens
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERNLSHNRRVPKIGPYPDYSSSSYIEDSIHHTDSIVKNISSEHSMSSTPLTIGEKNRNSINYERQQAQARIPSPETSVTSLSTNTTTTNTTGLTPSTGMTTISEMPYPDETNLHTTNVAQSIGPTPVCLQLTEEDLETNKLDPKEVDKNLKESSDENLMEHSLKQFSGPDPLSSTSSSLLYPLIKLAVEATGQQDFTQTANGQACLIPDVLPTQIYPLPKQQNLPKRPTSLPLNTKNSTKEPRLKFGSKHKSNLKQVETGVAKMNTINAAEPHVVTVTMNGVAGRNHSVNSHAATTQYANGTVLSGQTTNIVTHRAQEMLQNQFIGEDTRLNINSSPDEHEPLLRREQQAGHDEGVLDRLVDRRERPLEGGRTNSNNNNSNPCSEQDVLAQGVPSTAADPGPSKPRRAQRPNSLDLSATNVLDGSSIQIGESTQDGKSGSGEKIKKRVKTPYSLKRWRPSTWVISTESLDCEVNNNGSNRAVHSKSSTAVYLAEGGTATTMVSKDIGMNCL

(19) United States
(12) Patent Application Publication
(10) Pub. No.: US 2009/0029371 A1
(43) Pub. Date: Jan. 29, 2009

(54) METHOD FOR DETERMINING VASOREACTIVITY

(75) Inventor: C. Gregory Elliott, Salt Lake City, UT (US)

Correspondence Address: BRINKS HOFER GILSON & LIONE/UTAH;UTAH OFFICE, 405 South Main Street, Suite 800, SALT LAKE CITY, UT, 84111-3400, US

(73) Assignee: IHC intellectual Asset Management, LLC, Salt Lake City, UT (US)

(21) Appl. No.: 12/096,043

(22) PCT Filed: Dec. 5, 2006
(86) PCT No.: PCT/US06/61618

§ 371 Date: Jun. 4, 2008

Related U.S. Application Data
(60) Provisional application No. 60/742,454, filed on Dec. 5, 2005.
(60) Provisional application No. 60/747,073, filed on May 11, 2006.

(51) Int. Cl. C12Q 1/68; C07H 21/04; C07K 16/18

(52) U.S. Cl. 435/6; 536/23.5; 530/389.1

(57) ABSTRACT

The present invention is generally directed to methods and materials for determining the genotype of a patient to predict the patient's vasoreactivity. More particularly, the present invention is directed to a method of determining the vasoreactivity of a subject, comprising: obtaining from a subject a sample comprising a nucleic acid sequence of the BMPR2 gene or amino acid sequence of the BMPR2 gene; determining the presence or absence of a non-synonymous mutation in the BMPR2 nucleic acid or amino acid sequence, and correlating the presence of a non-synonymous mutation with non-vasoreactivity or the absence of a non-synonymous mutation with vasoreactivity.

### CROSS REFERENCE TO RELATED APPLICATION

This application claims the benefit of the filing date of U.S. Provisional Patent Application Ser. No. 60/742,454, filed Dec. 5, 2005, and of U.S. Provisional Patent Application Ser. No. 60/747,073, filed May 11, 2006, the disclosures of which are incorporated, in their entirety, by this reference.

### FIELD OF INVENTION

The present invention relates to the field of pharmacogenomics, and more particularly to methods for determining a genotype that is predictive of vasoreactivity.

### BACKGROUND OF INVENTION

Idiopathic pulmonary arterial hypertension is a progressive disorder characterized by a sustained abnormally high blood pressure (hypertension) in the arteries of the lungs (pulmonary arteries) without a demonstrable cause [Dresdale, 1951; Rubin, 2004]. Various factors are believed to contribute to increased pulmonary artery pressure and increased pulmonary vascular resistance, including vasoconstriction, thrombotic obstruction, or dysregulated cellular proliferation that obstructs the vascular lumen [Rubin, 1997; Wood, 1958]. Early reports of multiple family members affected by idiopathic pulmonary arterial hypertension suggested an inherited predisposition to this disorder [Dresdale, 1954].

Two groups of investigators have independently reported that mutations in the gene encoding bone morphogenetic protein receptor type-2 (BMRP2), a TGF P receptor, cause familial pulmonary arterial hypertension [Deng, 2000; Lane, 2000]. The BMPR2 gene belongs to a family of genes originally identified for its role in regulating the growth and maturation (differentiation) of bone and cartilage. The BMPR2 gene is located on the long (q) arm of chromosome 2, between positions 33 and 34 (2q33-q34). More precisely, the BMPR2 gene is located from base pair 203,067,104 to base pair 203,257,979 on chromosome 2.

Recently, researchers have found that the BMPR2 gene family plays a broader role in regulating growth and differentiation in numerous types of cells. By forming a complex with other proteins, BMPR2 plays an important role in regulating the number of cells in certain tissues. The BMPR2 protein is a receptor protein that spans the cell membrane, with one end of the protein extending from the outer surface of the cell and the other end remaining inside the cell. This arrangement allows the BMPR2 protein to receive and transmit signals that help the cell respond to its environment by growing and dividing (cell proliferation) or by undergoing a controlled cell death (apoptosis). This balance of cell division and cell death regulates the number of cells. Research studies suggest that the BMPR2 protein helps prevent the overgrowth of cells in blood vessels in the lungs and therefore plays a critical role in maintaining the normal function of these vessels.

Researchers have identified more than 40 BMPR2 mutations that can cause primary pulmonary hypertension. Many of these mutations introduce a stop signal that halts protein production prematurely, decreasing the amount of functional BMPR2 protein. Other mutations prevent the BMPR2 protein from reaching the cell surface, or alter its structure so it cannot form a complex with other proteins. It remains unclear how BMPR2 mutations cause primary pulmonary hypertension. Researchers suggest that a mutation in this gene prevents cell death or promotes cell proliferation, resulting in an overgrowth of cells in the blood vessels of the lungs. Cell overgrowth can narrow the diameter of the vessels, restricting blood flow and resulting in elevated blood pressure.

BMPR2 mutations have been identified in 11-40% of idiopathic pulmonary arterial hypertension patients [Newman, 2004; Thomson, 2000; Morisaki, 2004; Koehler, 2004]. This condition is inherited in an autosomal dominant pattern, which means one copy of the altered gene is sufficient to cause the disorder. BMPR2 mutations may be spontaneous or inherited from a parent who does not express the disease. In some cases, the altered BMPR2 gene is inherited from an affected parent or a parent with an altered gene who does not develop primary pulmonary hypertension. These inherited cases are known as familial primary pulmonary hypertension. As the altered gene is passed down from one generation to the next, the disorder generally begins earlier in life and has more severe symptoms, a phenomenon referred to as anticipation. Most cases of primary pulmonary hypertension, however, occur in individuals who have no previous family history of the disorder. These new cases are known as sporadic primary pulmonary hypertension. Some of the sporadic cases are due to mutations in the BMPR2, but for many cases a gene mutation has not yet been identified. Current evidence suggests that BMPR2 likely regulates cellular proliferation rather than vasoconstriction. However, the histopathological and clinical features of familial pulmonary arterial hypertension are identical to those of non-familial idiopathic pulmonary arterial hypertension [Rubin, 1997; Loyd, 1988].

This gene encodes a member of the bone morphogenetic protein (BMP) receptor family of transmembrane serine/threonine kinases. The ligands of this receptor are BMPs, which are members of the TGF-beta superfamily. BMPs are involved in endochondral bone formation and embryogenesis. These proteins transduce their signals through the formation of heteromeric complexes of 2 different types of serine (threonine) kinase receptors: type I receptors of about 50-55 kD and type II receptors of about 70-80 kD. Type II receptors bind ligands in the absence of type I receptors, but they require their respective type I receptors for signaling, whereas type I receptors require their respective type II receptors for ligand binding. Mutations in this gene have been associated with primary pulmonary hypertension.

Patients with marked vasoreactivity respond favorably to treatment with vasodilators, especially calcium channel blockers [Rich, 1992; Raffy, 1996]. Calcium channel blockers are recommended as initial therapy for patients who respond acutely to vasodilators [Badesch, 2004; Galie, 2004]. Recent international consensus identifies vasoreactivity testing as a critical clinical step in the management of patients with idiopathic pulmonary arterial hypertension [Badesch, 2004; Galie, 2004], and current guidelines recommend testing of vasoreactivity for patients with pulmonary arterial hypertension.

In view of the clinical importance of diagnosing vasoreactivity in patients with pulmonary arterial hypertension for purposes of determining appropriate clinical intervention, improved methods of predicting vasoreactivity are desirable.

### SUMMARY OF INVENTION

The present invention is generally directed to methods and materials for determining the genotype of a patient to predict the patient's vasoreactivity. While genetic polymorphisms in the TGF-β type II receptor gene, BMPR2, have been known to predispose patients to develop pulmonary hypertension, the correlation between BMPR2 mutations and vasoreactivity has not previously been understood.

In one embodiment, the present invention is directed to a method of determining the vasoreactivity of a subject, comprising: obtaining from a subject a sample comprising a nucleic acid sequence of the BMPR2 gene or amino acid sequence of the BMPR2 gene; determining the presence or absence of a non-synonymous mutation in the BMPR2 nucleic acid or amino acid sequence, and correlating the presence of a non-synonymous mutation with non-vasoreactivity or the absence of a non-synonymous mutation with vasoreactivity.

The non-synonymous mutations in the BMPR2 nucleic acid or amino acid sequence corresponds to a mutation at any one or more of the following nucleotide positions of SEQ ID NO:1: 218, 354, 355, 367, 439, 504, 689, 958, 994, 1042, 1076, 1129, 1191, 1258, 1454, 1535, 1557, 1749, 2292, 2408, 2579, and 2695. More particularly, the non-synonymous mutation in the BMPR2 nucleic acid sequence or amino acid sequence corresponds to a mutation characterized as any one or more of the following, or a complement thereof: C218G, T354G, T367C, T367A, C439T, C994T, G1042A, T1258C, A1454G, A1535C, T1557A, C2695T.

The non-synonymous BMPR2 mutations in the BMPR2 nucleic acid sequence or amino acid sequence may also correspond to a mutation at any one or more of the following amino acid positions of SEQ ID NO:2: 73, 118, 123, 143, 332, 348, 420, 485, 512, 519, and 899. More particularly, the non-synonymous mutations in the BMPR2 nucleic acid sequence or amino acid sequence may correspond to a mutation characterized as any one or more of the following: 73term, 118W, 123R, 123S, 143term, 332term, 348I, 420R, 485A, 512GQterm, 519K, 899term.

The present invention is also directed to a method of treating a patient diagnosed with pulmonary arterial hypertension, comprising: obtaining from a subject a sample comprising a nucleic acid sequence of the BMPR2 gene or amino acid sequence of the BMPR2 gene; determining the presence or absence of a non-synonymous mutation in the BMPR2 nucleic acid or amino acid sequence; correlating the presence of a non-synonymous mutation with non-vasoreactivity or the absence of a non-synonymous mutation with vasoreactivity; and making a clinical decision whether to administer to the patient a therapeutic compound capable of eliciting a vasoresponse. The non-synonymous mutations may be any one of those described above.

In yet another embodiment, the present invention may be an isolated polynucleotide comprising a sequence of nucleic acids containing a polymorphism selected from the group consisting of: 188-208del121, G203, A, T295C, A600C, 968_{—}969insT, 1113_{—}1114insT, C1469T, and 2527delG. In still another embodiment, the present invention is directed to an antibody having specificity to any one of these mutations.

In still another embodiment, the present invention is directed to a kit for determining the vasoreactivity of a subject, comprising reagents for detecting the presence or absence of a non-synonymous mutation in the BMPR2 nucleic acid or BMPR2 protein of the subject.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF FIGURES

FIG. 1 is a table showing characteristics of twenty-nine patients with non-synonymous BMPR2 sequence variations.

### DETAILED DESCRIPTION OF INVENTION

### Definitions

Units, prefixes, and symbols may be denoted in their SI accepted form. Unless otherwise indicated, nucleic acids are written left to right in 5′ to 3′ orientation. Numeric ranges recited herein are inclusive of the numbers defining the range and include, and are supportive of, each integer within the defined range. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes. Unless otherwise noted, the singular form of such terms as “a,” “an,” and “the” are to be construed to include plural form, unless the context clearly dictates otherwise. Thus, for example, reference to “a host cell” includes a plurality of such host cells, reference to “the antibody” is a reference to one or more antibodies and equivalents thereof known to those skilled in the art, and so forth.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. In the case of any amino acid or nucleic sequence discrepancy within the application, the FIGURES control.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

The practice of the present invention will employ, unless otherwise indicated, conventional techniques of molecular biology, microbiology, recombinant DNA techniques, and nucleic acid synthesis, which are within the skill of the art. Such techniques are explained fully in the literature. The foregoing techniques and procedures are generally performed according to conventional methods well known in the art and as described in various general and more specific references that are cited and discussed throughout the present specification. See, e.g., Sambrook et al. Molecular Cloning: A Laboratory Manual (2d ed., Cold Spring Harbor Laboratory Press, Cold Spring Harbor, N.Y. (1989)); Nucleic acid Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.), the contents of all of which are incorporated herein by reference.

As utilized in accordance with the present disclosure, the following terms, unless otherwise indicated, shall be understood to have the following meanings:

“BMPR2” means the gene encoding TGF-beta type II receptor, also referred to as Bone Morphogenic Protein Receptor 2, as previously described in the art, or the protein encoded by this gene. As used herein, the term BMPR2 refers to both the BMPR2 gene and the polypeptide encoded by a BMPR2 gene. Both of these terms are used herein as general identifiers. Thus, for example, a BMPR2 gene or nucleic acid refers to any gene or nucleic acid identified with or derived from a wild-type BMPR2 gene. For example, a mutant BMPR2 gene is a form of the BMPR2 gene. The nucleotide sequence for the BMPR2 gene and its corresponding protein sequence are set forth in GenBank accession number NM_{—}001204. The coding sequence of the BMPR2 gene is disclosed herein in SEQ ID NO:1 and the protein translation product of the BMPR2 gene is disclosed in SEQ ID NO:2.

The term “corresponds,” as used in reference to a mutation in relation to a nucleic acid or amino acid sequence, means that the mutation is characterized or described by the mutation nomenclature, is located at the position identified, or is related by the biological process of transcription and translation of a nucleic acid codon (comprising three nucleotides) to an amino acid. A mutation in a nucleic acid sequence or amino acid sequence therefore “corresponds” to a particular position or locus of the respective nucleic acid sequence or amino acid sequence. A mutation in a nucleic acid sequence or amino acid sequence also “corresponds” to a particular nucleic acid sequence or amino acid sequence characterized by a nomenclature that identifies the position of the mutation and the identity of the new nucleic acid or amino acid that replaces the old nucleic acid or amino acid. The term “corresponds” is also used to describe a mutation in terms of the relationship between the mutation in the nucleic acid sequence and the resulting mutation in the amino acid sequence encoded by the nucleic acid sequence. For example, a mutation in a nucleic acid sequence “corresponds” to a mutation in an amino acid sequence that is encoded by the nucleic acid. Similarly, a mutation in an amino acid sequence “corresponds” to a mutation in a nucleic acid sequence that encodes the amino acid sequence.

“Isolated polypeptide” or “purified polypeptide” mean a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

“Isolated nucleic acid” or “purified nucleic acid” mean DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

The terms “nucleic acid,” “polynucleotide,” “oligonucleotide,” and “DNA,” refer to primers, probes, oligomer fragments to be detected, oligomer controls, unlabeled blocking oligomers and templates, and mean polydeoxyribonucleotides (containing 2-deoxy-D-ribose), polyribonucleotides (containing D-ribose), and any other type of polynucleotide which is an N-glycoside of a purine or pyrimidine base, or modified purine or pyrimidine bases. There is no intended distinction in length between the term “DNA,” “nucleic acid”, “polynucleotide” and “nucleic acid”, and these terms are used interchangeably herein. These terms refer only to the primary structure of the molecule. Thus, these terms include double- and single-stranded DNA, as well as double- and single-stranded RNA.

The nucleic acid sequences of the invention are not necessarily physically derived from any existing or natural sequence but may be generated in any manner, including chemical synthesis, DNA replication, reverse transcription or a combination thereof. The term “nucleic acid” may refer to a polynucleotide of genomic DNA or RNA, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature; and (3) is not found in nature.

Because mononucleotides are reacted to make nucleic acids in a manner such that the 5′ phosphate of one mononucleotide pentose ring is attached to the 3′ oxygen of its neighbor in one direction via a phosphodiester linkage, an end of a nucleic acid is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger nucleic acid, also may be said to have 5′ and 3′ ends.

The term “mutation” means a polymorphism or variant form of the nucleotide sequence of a DNA molecule, representing an alternative form of the DNA molecule. A mutation may occur in the form of a substitution of one nucleotide or region of polynucleotides for another nucleotide or region of polynucleotides, resulting in no net change in the number of nucleotides. Alternatively, a mutation may occur in the form of a deletion or insertion of one or more nucleotides or region of polynucleotides, resulting in a change in the number of nucleotides. While the term “mutation” is frequently used to refer to a particular variant different from a common or wild-type form of a DNA molecule (i.e., a variant that is present at a lower frequency relative to the population of organisms to which the variant relates), the term “mutation” is used herein to refer to any variant, including the common or wild-type variant, as well as variants present at lower frequencies. Further, the term “mutation” may refer to either a particular variant of a nucleotide sequence (an “allele”), or to any one of various mutations associated with a particular locus of a nucleotide sequence. Thus, reference to the mutations at a particular locus means that the nucleotide sequence of one chromosome of a particular individual is different from the nucleotide sequence of the other chromosome of the same individual or is different from the nucleotide sequence of a chromosome of another individual. Mutations may either be benign or causative of a particular phenotypic trait, such as a mutation that gives rise to a disease condition.

The term “non-synonymous mutation” means a mutation that results in a change in a codon of a reference gene such that the mutated codon encodes a different amino acid in the protein encoded by the reference gene. Conversely, a synonymous mutation is a mutation in a codon of a reference gene which, due to the degeneracy of the genetic code, results in the same amino acid in the protein encoded by the reference gene. A synonymous mutation is also commonly referred to as a “silent” mutation, since it does not result in a change in amino acid that it encodes.

The term “primer” means a defined polynucleotide fragment that is capably of hybridizing to a complementary nucleic acid template to form a double stranded complex, due to complementarity of nucleotide sequence in the probe with a nucleotide sequence in the template, and initiating synthesis of a second strand of nucleic acid in the presence of a polymerase enzyme and other nucleotide feedstocks.

The term “probe” means a defined polynucleotide fragment that is capable of hybridizing to a complementary nucleic acid template to form a double stranded complex, due to complementarity of nucleotide sequence in the probe with a nucleotide sequence in the template. A probe typically contains a detectable radioactive or chemical label enabling detection of the probe by any of various means known to those in the art. As used herein, the term “probe” specifically refers to a polynucleotide fragment that is blocked at the 3′ end, for example, with a 2′-,3′-dideoxynucleotide, with a phosphate group, or with any other chemical moiety that blocks or removes free 3′ hydroxyl group necessary for primer extension.

The term “pulmonary arterial hypertension” means a disorder characterized by a sustained abnormally high blood pressure (hypertension) in the arteries of the lungs.

The term “sample,” as used herein, means a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components, such as a nucleic acid.

The term “vasoreactive” means that a subject responds acutely to a vasodilator compound. The term “vasoreactivity” refers to the degree to which a patient responds to a vasodilator compound.

The publications and other materials used herein to illustrate the background of the invention, and in particular, cases to provide additional details respecting the practice, are incorporated herein by reference, and for convenience, are referenced by author and date in the following text and respectively grouped in the appended List of References.

The present invention is based on the discovery that patients with idiopathic or familial pulmonary arterial hypertension and BMPR2 mutations are unlikely to respond acutely to vasodilators (i.e., to demonstrate vasoreactivity). More particularly, the present invention relates to the surprising discovery that mutations in the gene encoding a TGF-beta type II receptor (BMPR2) gene are predictive of vasoreactivity (i.e., the response of an individual to a therapeutic compound capable of modulating the vasoresponse). This observation has potentially significant clinical implications. Based on this observation, the present invention provides methods and materials for the diagnosis of vasoreactivity and subsequent clinical intervention.

The present invention is generally directed to a method of determining the vasoreactivity of a subject, comprising the steps of (1) obtaining from a subject a sample comprising a nucleic acid sequence of the BMPR2 gene or amino acid sequence of the BMPR2 gene, and (2) determining the presence or absence of a non-synonymous mutation in the BMPR2 nucleic acid or amino acid sequence, and (3) correlating the presence of a non-synonymous mutation with non-vasoreactivity or the absence of a non-synonymous mutation with vasoreactivity. The present invention is also directed to a method of treating a patient diagnosed with pulmonary arterial hypertension, comprising the steps of (1) obtaining from a subject a sample comprising a nucleic acid sequence of the BMPR2 gene or amino acid sequence of the BMPR2 gene; (2) determining the presence or absence of a non-synonymous mutation in the BMPR2 nucleic acid or amino acid sequence, (3) correlating the presence of a non-synonymous mutation with non-vasoreactivity or the absence of a non-synonymous mutation with vasoreactivity, and (4) making a clinical decision whether to administer to the patient a therapeutic compound capable of eliciting a vasoresponse.

The present invention also provides novel BMPR2 mutations that are predictive of the vasoreactivity of a subject, nucleic acids that comprise such novel BMPR2 mutations, and antibodies that specifically bind to the corresponding mutations in the BMPR2 protein.

As set forth herein, nucleotides are numbered according to the cDNA sequence for BMPR2 (SEQ ID NO:1), with the adenosine of the initiation codon assigned position 1. (Kawabata, M., Chytil, A. & Moses, H. L., “Cloning of a novel type II serine/threonine kinase receptor through interaction with the type I transforming growth factor-beta receptor,” J. Biol. Chem. 270, 5625-5630 (1995); Liu, F., Ventura, F., Doody, J. & Massagu, J. “Human type II receptor for bone morphogenic proteins (BMPs): Extension of the two-kinase receptor model to the BMPs,” Mol. C ell. Biol. 15, 3479-3486 (1995); Rosenzweig, B. L. et al., “Cloning and characterization of a human type II receptor for bone morphogenetic proteins,” Proc. Natl. Acad. Sci. U.S.A. 92, 7632-7636 (1995).

The nucleotide sequence, and corresponding amino acid translation product, of the coding region of BMPR2 are shown in SEQ ID NO:1 and SEQ ID NO:2, respectively. The sequence of SEQ ID NO:1 and SEQ ID NO:2 are derived from GenBank Accession No. NM 001204. SEQ ID NO:1 shows the nucleotide sequence of the entire coding sequence (CDS) as nucleotides 1-3114, with the adenosine of the initiation codon designated as nucleotide 1. The signal peptide consists of nucleotides 1-78, and the mature protein consists of nucleotides 79-3114. SEQ ID NO:2 shows the entire translation of the above CDS as amino acids 1-1038. The signal peptide consists of amino acids 1-26, and mature protein consists of amino acids 27-1038.

The nomenclature used herein to describe and define mutations is as follows. All notations referencing a nucleotide or amino acid residue will be understood to correspond to the residue number of the wild-type BMPR2 nucleic acid sequence set forth at SEQ ID NO:1, or of the wild-type BMPR2 polypeptide sequence set forth at SEQ ID NO:2. Thus, for example, the notation “T367C” indicates that the nucleotide T at position 367 of the sequence set forth at SEQ ID NO:1 has been replaced with a C. Similarly, the notation “355deIA” indicates that the nucleotide A at position 355 has been deleted. Furthermore, the notation 2408insTG” indicates that the nucleotides T and G, in that order, have been inserted following the nucleotide at position 2408. Similarly, mutations in the amino acid sequence are described using nomenclature that identifies the affected amino acid, followed by the amino acid residue or position number, and a brief description of the type of mutation and the number of amino acids affects. For example D323fsX3 denotes that the D (or Asp) amino acid at position 323 has resulted in a frame shift (fs) mutation that results in a change of 3 amino acids. The nomenclature 899term means that a mutation occurs N-terminal to amino acid 899, resulting in premature termination of the nucleic acid and protein. The nomenclature used herein is standard nomenclature understood and used by those skilled in the art.

In the method of the invention, the non-synonymous mutations in the BMPR2 polypeptide or BMPR2 nucleic acid are associated with non-vasoreactivity. Thus, the presence of a non-synonymous mutation in the BMPR2 polypeptide or BMPR2 nucleic acid indicates that the patient is vasoreactive and would be predicted to be responsive to vasodilators. Conversely, the absence of a non-synonymous mutation in the BMPR2 polypeptide or BMPR2 nucleic acid is indicative that the patient is not vasoreactive and would not be predicted to be responsive to vasodilators. Accordingly, the presence of non-synonymous BMPR2 mutations predicts that the subject will not respond to vasodilators, and avoids the necessity of independent vasoreactivity testing. Similarly, the absence of non-synonymous BMPR2 mutations predicts that the subject will respond to vasodilators. In either case, the vasoreactivity status of the patient can be used to make clinical decisions regarding appropriate treatment of the patient, and to treat the patient accordingly. The vasoreactivity status of a subject can therefore be determined more cost effectively and more expeditiously using standard genotyping protocols.

The present invention specifically provides for a method of characterizing BMPR2 mutations and identifying patients who will not respond to vasodilators acutely and who are therefore unlikely to benefit from prolonged treatment with vasodilators such as calcium channel blockers. In addition, the present invention provides for a method of characterizing BMPR2 mutations and identifying patients who will respond to vasodilators acutely and who are therefore likely to benefit from prolonged treatment with vasodilators.

In particular embodiments, the present invention is directed to a method of determining the vasoreactivity of a subject, comprising: obtaining from a subject a sample comprising a nucleic acid sequence of the BMPR2 gene or amino acid sequence of the BMPR2 gene; determining the presence or absence of a non-synonymous mutation in the BMPR2 nucleic acid or amino acid sequence, and correlating the presence of a non-synonymous mutation with non-vasoreactivity or the absence of a non-synonymous mutation with vasoreactivity.

Certain non-synonymous mutations in the BMPR2 gene (and the corresponding mutation in the BMPR2 protein) are already well-known in the art. The non-synonymous mutations in the BMPR2 nucleic acid or amino acid sequence may, for example, correspond to a mutation at any one or more of the following nucleotide positions of SEQ ID NO:1: 218, 354, 355, 367, 439, 504, 689, 958, 994, 1042, 1076, 1129, 1191, 1258, 1454, 1535, 1557, 1749, 2292, 2408, 2579, and 2695. Specific, non-synonymous mutation in the BMPR2 nucleic acid sequence or amino acid sequence may correspond to a mutation characterized as any one or more of the following, or a complement thereof: C218G, T354G, T367C, T367A, C439T, C994T, G1042A, T1258C, A1454G, A1535C, T1557A, C2695T. The above-identified non-synonymous mutations are provided by way of example, not by way of limitation. It is understood that the methods and materials of the present invention are applicable to all non-synonymous mutations.

The non-synonymous BMPR2 mutations in the BMPR2 nucleic acid sequence or amino acid sequence may also correspond to a mutation at any one or more of the following amino acid positions of SEQ ID NO:2: 73, 118, 123, 143, 332, 348, 420, 485, 512, 519, and 899. More particularly, the non-synonymous mutations in the BMPR2 nucleic acid sequence or amino acid sequence may correspond to a mutation characterized as any one or more of the following: 73term, 118W, 123R, 123S, 143term, 332term, 348I, 420R, 485A, 512GQterm, 519K, 899term.

The present invention is also directed to a method of treating a patient diagnosed with pulmonary arterial hypertension, comprising: obtaining from a subject a sample comprising a nucleic acid sequence of the BMPR2 gene or amino acid sequence of the BMPR2 gene; determining the presence or absence of a non-synonymous mutation in the BMPR2 nucleic acid or amino acid sequence; correlating the presence of a non-synonymous mutation with non-vasoreactivity or the absence of a non-synonymous mutation with vasoreactivity; and making a clinical decision whether to administer to the patient a therapeutic compound capable of eliciting a vasoresponse. The non-synonymous mutations may be any one of those described above.

In another embodiment, the present invention is directed to novel mutations in the BMPR2 nucleic acid or protein. The present invention therefore provides an isolated polynucleotide comprising a sequence of nucleic acids containing a polymorphism selected from the group consisting of: 188-208del121, G203, A, T295C, A600C, 968_{—}969insT, 1113_{—}1114insT, C1469T, and 2527delG.

The invention also provides an antibody having specificity to any one of the following mutations: 188-208del121, G203, A, T295C, A600C, 968_{—}969insT 1113_{—}1114insT, C1469T, and 2527delG.

In still another embodiment, the present invention is directed to a kit for determining the vasoreactivity of a subject, comprising reagents for detecting the presence or absence of a non-synonymous mutation in the BMPR2 nucleic acid or BMPR2 protein of the subject.

In a study, the individuals with variant DNA who demonstrated vasoreactivity had synonymous BMPR2 mutations. Thus the detection of non-synonymous mutations is predictive of patients who do not respond acutely to vasodilators. Conversely, an acute vasodilator response may occur when tests find synonymous mutations or common polymorphisms in the BMPR2 gene. These observations may represent a step toward individualized treatment for patients with idiopathic and familial pulmonary arterial hypertension based upon genetic tests. Genotyping patients to detect the presence or absence of synonymous or non-synonymous BMPR2 mutations (i.e., mutation that alter the amino acid sequence and protein product) can be useful in determining the appropriate course of therapeutic treatment, without the need for performing costly and possibly harmful vasoreactivity tests. Current evidence suggests that at least half of all familial pulmonary arterial hypertension patients [Cogan, 2005] and approximately 11 to 40 percent of idiopathic pulmonary arterial hypertension patients [Thomson, 2000; Koehler 2004; Morisaki, 2004] might avoid the costs and the rare but serious risk of death during vasoreactivity tests [Weir 1989; Ricciardi, 2001].

While not being bound by any particular theory, the discovery upon which the present invention is based is consistent with the hypothesis that dysregulated cellular proliferation underlies pulmonary arterial hypertension accompanied by BMPR2 mutations [Loscalzo, 2001]. This discovery is also consistent with the pathologic findings of laminar intimal fibrosis, plexiform lesions with obstruction of the pulmonary artery lumen and proliferation of endothelial cells [Loyd 1988; Runo, 2003; Lee, 1998]. Dysfunctional BMPR2 may convert smooth muscle or endothelial cells from an antiproliferative to a proliferative phenotype [McCaffrey, 1995]. Furthermore, this discovery is also consistent with the recent observation that loss of BMPR2 signaling in transgenic mice causes pulmonary arterial hypertension [West, 2005].

In one embodiment, subjects having an increased susceptibility for developing pulmonary hypertension can be identified by detecting the presence or absence of a mutation in the BMPR2 nucleic acid in the subject. In another embodiment, subjects having an increased susceptibility for developing pulmonary hypertension can be identified by detecting the presence or absence of a non-synonymous mutation in the BMPR2 nucleic acid in the subject. Mutations in the BMPR2 nucleic acid can be detected directly, by analyzing the nucleic acid directly, or can be detected indirectly, such as by detecting mutations in the BMPR2 protein, from which one can of course infer that a nucleic acid mutation is causally responsible for the amino acid mutation.

The mutated BMPR2 nucleic acid may comprise a missense mutation, that is, a mutation that changes a codon specific for one amino acid to a codon specific for another amino acid. Examples of mutated BMPR2 nucleic acids having a missense mutation which is associated with pulmonary hypertension include C218G, T354G, T367C, T367A, C439T, C994T, G1042A, T1258C, A1454G, A1535C, T1557A, and C2695T.

In another embodiment, the BMPR2 nucleic acid having a sequence associated with pulmonary hypertension comprises a nucleic acid sequence having an insertion mutation, where one or more nucleotides are inserted into the wild-type sequence.

The mutated BMPR2 nucleic acid may also comprise a deletion mutation, where one or more nucleotides are deleted from the wild-type sequence. Such a deletion or insertion mutation may, for example, result in a frameshift mutation, altering the reading frame. Frameshift mutations typically result in truncated or prematurely terminated BMPR2 polypeptide. Examples of BMPR2 nucleic acids having an insertion mutation which are associated with pulmonary hypertension include 504insT, 2292insA, and 2408insTG. Examples of BMPR2 nucleic acids having a deletion mutation which are associated with pulmonary hypertension include 355delA, 689delA, 958delT, 1076delC, 1191/1192delTG, and 2579delT.

The mutated BMPR2 nucleic acid may also comprise a nonsense mutation, that is, a mutation that changes a codon specific for an amino acid to a chain termination codon. Nonsense mutations result in truncated or prematurely terminated BMPR2 polypeptides. Examples of BMPR2 nucleic acids having a nonsense mutation which are associated with pulmonary hypertension include C218G, C439T, C994T, and C2695T.

The mutated BMPR2 nucleic acid may also comprise a truncation mutation, that is, a mutated BMPR2 nucleic acid which encodes a truncated BMPR-II polypeptide. This may occur where, for example, the BMPR2 nucleic acid has a nonsense mutation. In another embodiment, the mutated BMPR2 nucleic acid can be truncated at a nucleotide position of the sequence set forth in SEQ ID NO:1 which is 3′ to nucleotide position 2695 of the sequence set forth at SEQ ID NO:1. A mutation at nucleotide 2695, which truncates the BMPR2 polypeptide at amino acid residue 899, is also expected to be indicative of vasoreactivity.

Examples of non-synonymous mutations in the nucleic acid sequence of BMPR2 include, for example, mutations at the following nucleotide position of the sequence set forth in SEQ ID NO:1: 218, 354, 355, 367, 439, 504, 689, 958, 994, 1042, 1076, 1129, 1191, 1258, 1454; 1535, 1557, 1749, 2292, 2408, and 2695. Other known non-synonymous mutations include mutations at the following nucleotide positions of SEQ ID NO:1: 185-208, 203, 295, 350, 439, 631, 637, 727, 968-969, 994, 1113-1114, 1114-1115, 1129-3, 1248, 1397, 1469, 2527, 2579, 2617. The mutation can result in a polypeptide having a non-conservative substitution at the relevant amino acid residue. One of ordinary skill will readily understand the concept of a “non-conservative substitution.” Substitutions such as a charged amino acid for an uncharged amino acid, or an uncharged amino acid for a charged amino acid, or any amino acid in place of a Cys, or visa versa, or any amino acid in place of a Pro, or visa versa, are well known in the art to alter the structure and often the function of a protein. The mutation can also result in reduction or elimination of BMPR2 mRNA production, incorrect or altered processing of BMPR2 RNA, increased BMPR2 RNA instability, or other effects on expression of BMPR2 prior to translation. For example, the mutation 1129CG alters a splice junction and results in incorrect splicing of BMPR2 RNA. The mutation C1749T, which does not alter the encoded amino acid, likely affects RNA production, processing, or function.

Non-synonymous mutations may comprise one or more mutations in the amino acid sequence of SEQ ID NO:2. In the embodiment wherein the mutation in the mutated BMPR2 nucleic acid results in a non-synonymous substitution in the amino acid sequence encoded by the nucleic acid, the mutation in the mutated BMPR2 nucleic acid can be selected from one or more of the following: 185-208del21, G203A, C218G, T295C, G350A transition, C439T, A600C, C631T, C637T, G727T, 968-969insT, C994T, 1113-1114insT, 1114-1115insT, C1129-3G (splice site mutation in intron 8), 1248delA, G1397A, C1469T, 2527delG, 2579delG, and C2617T. Other possible mutations in the BMPR2 nucleic acid include the following: T354G, 355, T367C, T367A, C439T, 504insT, 689delA, 958delT, C994T, G1042A, 1076delC, 1191/1192delTG, T1258C, A1454G, A1535C, T1557A, C1749T, 2292insA, 2408insTG, 2579delT, C2695T.

In one aspect, the invention is directed to probes and primers for use in a prognostic or diagnostic assay, comprising the novel mutations described above. For instance, the present invention also provides a probe/primer comprising a substantially purified oligonucleotide, which oligonucleotide comprises a region of nucleotide sequence that hybridizes under stringent conditions to at least approximately 12, preferably 25, more preferably 40, 50 or 75 consecutive nucleotides of sense or anti-sense sequence of BMPR2, including 5′ and/or 3′ untranslated regions. In preferred embodiments, the probe further comprises a label group attached thereto and able to be detected, e.g. the label group is selected from amongst radioisotopes, fluorescent compounds, enzymes, and enzyme co-factors.

In a further aspect, the present invention features methods for determining whether a subject is vasoreactive (i.e., responsive to vasodilators). According to the diagnostic and prognostic methods of the present invention, alteration of the wild-type BMPR2 locus that result in a non-synonymous mutation is detected. A non-synonymous mutations includes all forms of mutations including deletions, insertions and point mutations in the coding and noncoding regions that result in a change in the amino acid sequence of the wild-type BMPR2 protein. Thus, in a particular embodiment of the invention, the BMPR2 mutations detected are non-synonymous mutations, which alter the amino acid sequence of the wild-type BMPR2 gene and protein. Deletions may be of the entire gene or of only a portion of the gene. Point mutations may result in stop codons, frameshift mutations or amino acid substitutions. Point mutations or deletions in the promoter can change transcription and thereby alter the gene function. Somatic mutations are those which occur only in certain tissues and are not inherited in the germline. Germline mutations can be found in any of a body's tissues and are inherited. The finding of BMPR2 germline mutations thus provides diagnostic information. A BMPR2 allele which is not deleted (e.g., found on the sister chromosome to a chromosome carrying an BMPR2 deletion) can be screened for other mutations, such as insertions, small deletions, and point mutations. Point mutational events may occur in regulatory regions, such as in the promoter of the gene, or in intron regions or at intron/exon junctions.

Diagnostic techniques that are useful for identifying BMPR2 mutations include, but are not limited to the following: fluorescent in situ hybridization (FISH), direct DNA sequencing, PFGE analysis, Southern blot analysis, single stranded conformation analysis (SSCA), RNase protection assay, allele-specific oligonucleotide (ASO), and dot blot analysis and PCR-SSCP, and mass spectrometry, several of which are described in more detail below. Also useful is the recently developed technique of DNA microchip technology. In addition to the techniques described herein, similar and other useful techniques are also described in U.S. Pat. Nos. 5,837,492 and 5,800,998, each incorporated herein by reference.

Vasoreactivity can be ascertained by testing any tissue of a human for mutations of the BMPR2 gene. For example, a person who has inherited a germline BMPR2 mutation would be prone to being non-vasoreactive. This can be determined by testing DNA from any tissue of the person's body. Most simply, blood can be drawn and DNA extracted from the cells of the blood. Alteration of a wild-type BMPR2 allele, whether, for example, by point mutation or deletion, can be detected by any of the means discussed herein.

There are several methods that can be used to detect DNA sequence variation. Direct DNA sequencing, either manual sequencing or automated fluorescent sequencing can detect sequence variation. Manual sequencing can be very labor-intensive, but under optimal conditions, mutations in the coding sequence of a gene are rarely missed. Another approach is the single-stranded conformation polymorphism assay (SSCA) (Orita et al., 1989). This method does not detect all sequence changes, especially if the DNA fragment size is greater than 200 bp, but can be optimized to detect most DNA sequence variation. The reduced detection sensitivity is a disadvantage, but the increased throughput possible with SSCA makes it an attractive, viable alternative to direct sequencing for mutation detection on a research basis. The fragments which have shifted mobility on SSCA gels are then sequenced to determine the exact nature of the DNA sequence variation. Other approaches based on the detection of mismatches between the two complementary DNA strands include clamped denaturing gel electrophoresis (CDGE) (Sheffield et al., 1991), heteroduplex analysis (HA) (White et al., 1992) and chemical mismatch cleavage (CMC) (Grompe et al., 1989). None of the methods described above will detect large deletions, duplications or insertions, nor will they detect a regulatory mutation which affects transcription or translation of the protein. Other methods which might detect these classes of mutations such as a protein truncation assay or the asymmetric assay, detect only specific types of mutations and would not detect missense mutations. A review of currently available methods of detecting DNA sequence variation can be found in a recent review by Grompe (1993). Once a mutation is known, an allele specific detection approach such as allele specific oligonucleotide (ASO) hybridization can be utilized to rapidly screen large numbers of other samples for that same mutation.

Detection of point mutations may be accomplished by molecular cloning of the BMPR2 allele(s) and sequencing the allele(s) using techniques well known in the art. Alternatively, the gene sequences can be amplified directly from a genomic DNA preparation from the tissue, using known techniques. The DNA sequence of the amplified sequences can then be determined.

Well known methods for a more complete, yet still indirect, test for confirming the presence of a mutant allele, include the following: 1) single-stranded conformation analysis (SSCA) (Orita et al., 1989); 2) denaturing gradient gel electrophoresis (DGGE) (Wartell et al., 1990; Sheffield et al., 1989); 3) RNase protection assays (Finkelstein et al., 1990; Kinszler et al., 1991); 4) allele-specific oligonucleotides (ASOs) (Conner et al., 1983); 5) the use of proteins which recognize nucleotide mismatches, such as the E. coli mutS protein (Modrich, 1991); and 6) allele-specific PCR (Rano and Kidd, 1989). For allele-specific PCR, primers are used which hybridize at their 3′ ends to a particular BMPR2 mutation. If the particular BMPR2 mutation is not present, an amplification product is not observed. Amplification Refractory Mutation System (ARMS) can also be used, as disclosed in European Patent Application Publication No. 0332435 and in Newton et al., 1989. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Such a method is particularly useful for screening relatives of an affected individual for the presence of the BMPR2 mutation found in that individual. Other techniques for detecting insertions and deletions as known in the art can be used.

In the first three methods (SSCA, DGGE and RNase protection assay), a new electrophoretic band appears. SSCA detects a band which migrates differentially because the sequence change causes a difference in single-strand, intramolecular base pairing. RNase protection involves cleavage of the mutant polynucleotide into two or more smaller fragments. DGGE detects differences in migration rates of mutant sequences compared to wild-type sequences, using a denaturing gradient gel. In an allele-specific oligonucleotide assay, an oligonucleotide is designed which detects a specific sequence, and the assay is performed by detecting the presence or absence of a hybridization signal. In the mutS assay, the protein binds only to sequences that contain a nucleotide mismatch in a heteroduplex between mutant and wild-type sequences.

Mismatches, according to the present invention, are hybridized nucleic acid duplexes in which the two strands are not 100% complementary. Lack of total homology may be due to deletions, insertions, inversions or substitutions. Mismatch detection can be used to detect point mutations in the gene or in its mRNA product. While these techniques are less sensitive than sequencing, they are simpler to perform on a large number of tumor samples. An example of a mismatch cleavage technique is the RNase protection method. In the practice of the present invention, the method involves the use of a labeled riboprobe which is complementary to the human wild-type AGT gene coding sequence. The riboprobe and either mRNA or DNA isolated from the tumor tissue are annealed hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full length duplex RNA for the riboprobe and the mRNA or DNA. The riboprobe need not be the full length of the BMPR2 mRNA or gene but can be a segment of either. If the riboprobe comprises only a segment of the BMPR2 mRNA or gene, it will be desirable to use a number of these probes to screen the whole mRNA sequence for mismatches.

In similar fashion, DNA probes can be used to detect mismatches, through enzymatic or chemical cleavage. See, e.g., Cotton et al., 1988; Shenk et al., 1975; Novack et al., 1986. Alternatively, mismatches can be detected by shifts in the electrophoretic mobility of mismatched duplexes relative to matched duplexes. See, e.g., Cariello, 1988. With either riboprobes or DNA probes, the cellular mRNA or DNA which might contain a mutation can be amplified using PCR before hybridization. Changes in DNA of the BMPR2 gene can also be detected using Southern hybridization, especially if the changes are gross rearrangements, such as deletions and insertions.

DNA sequences of the BMPR2 gene which have been amplified by use of PCR may also be screened using allele-specific probes. These probes are nucleic acid oligomers, each of which contains a region of the BMPR2 gene sequence harboring a known mutation. For example, one oligomer may be about 30 nucleotides in length (although shorter and longer oligomers are also usable as well recognized by those of skill in the art), corresponding to a portion of the BMPR2 gene sequence. By use of a battery of such allele-specific probes, PCR amplification products can be screened to identify the presence of a previously identified mutation in the BMPR2 gene. Hybridization of allele-specific probes with amplified BMPR2 sequences can be performed, for example, on a nylon filter. Hybridization to a particular probe under high stringency hybridization conditions indicates the presence of the same mutation in the tumor tissue as in the allele-specific probe.

The technique of nucleic acid analysis via microchip technology is also applicable to the present invention. In this technique, literally thousands of distinct oligonucleotide probes are built up in an array on a silicon chip. Nucleic acid to be analyzed is fluorescently labeled and hybridized to the probes on the chip. It is also possible to study nucleic acid-protein interactions using these nucleic acid microchips. Using this technique one can determine the presence of mutations or even sequence the nucleic acid being analyzed or one can measure expression levels of a gene of interest. The method is one of parallel processing of many, even thousands, of probes at once and can tremendously increase the rate of analysis. Several papers have been published which use this technique. Some of these are Hacia et al., 1996; Shoemaker et al., 1996; Chee et al., 1996; Lockhart et al., 1996; DeRisi et al., 1996; Lipshutz et al., 1995. This method has already been used to screen people for mutations in the breast cancer gene BRCA1 (Hacia et al., 1996). This new technology has been reviewed in a news article in Chemical and Engineering News (Borman, 1996) and been the subject of an editorial (Nature Genetics, 1996). Also see Fodor (1997).

The most definitive test for mutations in a candidate locus is to directly compare genomic BMPR2 sequences from disease patients with those from a control population. Alternatively, one could sequence messenger RNA after amplification, e.g., by PCR, thereby eliminating the necessity of determining the exon structure of the candidate gene.

Alteration of BMPR2 mRNA expression can be detected by any techniques known in the art. These include Northern blot analysis, PCR amplification and RNase protection. Diminished or increased mRNA expression indicates an alteration of the wild-type BMPR2 gene. Alteration of wild-type BMPR2 genes can also be detected by screening for alteration of wild-type BMPR2 protein. For example, monoclonal antibodies immunoreactive with BMPR2 can be used to screen a tissue. Lack of cognate antigen would indicate an BMPR2 mutation. Antibodies specific for products of mutant alleles could also be used to detect mutant BMPR2 gene product. Such immunological assays can be done in any convenient formats known in the art. These include Western blots, immunohistochemical assays and ELISA assays. Any means for detecting an altered BMPR2 protein can be used to detect alteration of wild-type BMPR2 genes. Functional assays, such as protein binding determinations, can be used. In addition, assays can be used which detect BMPR2 biochemical function. Finding a mutant BMPR2 gene product indicates alteration of a wild-type BMPR2 gene.

The primer pairs of the present invention are useful for determination of the nucleotide sequence of a particular BMPR2 allele using PCR. The pairs of single-stranded DNA primers can be annealed to sequences within or surrounding the BMPR2 gene in order to prime amplifying DNA synthesis of the BMPR2 gene itself. A complete set of these primers allows synthesis of all of the nucleotides of the BMPR2 gene coding sequences, i.e., the exons. The set of primers preferably allows synthesis of both intron and exon sequences. Allele-specific primers can also be used. Such primers anneal only to particular BMPR2 mutant alleles, and thus will only amplify a product in the presence of the mutant allele as a template.

In order to facilitate subsequent cloning of amplified sequences, primers may have restriction enzyme site sequences appended to their 5′ ends. Thus, all nucleotides of the primers are derived from BMPR2 sequences or sequences adjacent to BMPR2, except for the few nucleotides necessary to form a restriction enzyme site. Such enzymes and sites are well known in the art. The primers themselves can be synthesized using techniques which are well known in the art. Generally, the primers can be made using oligonucleotide synthesizing machines which are commercially available. Given the known sequences of the BMPR2 exons and the 5′ alternate exon, the design of particular primers is well within the skill of the art. Suitable primers for mutation screening are also described herein.

The nucleic acid probes provided by the present invention are useful for a number of purposes. They can be used in Southern hybridization to genomic DNA and in the RNase protection method for detecting point mutations already discussed above. The probes can be used to detect PCR amplification products. They may also be used to detect mismatches with the BMPR2 gene or mRNA using other techniques.

In accordance with the present invention, it has been discovered that individuals with the wild-type BMPR2 gene respond acutely to vasodilators. However, individuals with mutations in the BMPR2 gene do not respond acutely to vasodilators. Thus, the presence of an altered (or a mutant) BMPR2 gene directly correlates to an increase in vasoreactivity of an individual. In order to detect an BMPR2 gene mutation, a biological sample is prepared and analyzed for a difference between the sequence of the BMPR2 allele being analyzed and the sequence of the wild-type BMPR2 allele. Mutant BMPR2 alleles can be initially identified by any of the techniques described above. The mutant alleles are then sequenced to identify the specific mutation of the particular mutant allele. Alternatively, mutant BMPR2 alleles can be initially identified by identifying mutant (altered) BMPR2 proteins, using conventional techniques. The mutant alleles are then sequenced to identify the specific mutation for each allele. The mutations, especially non-synonymous mutations that result in a change in amino acid sequence, are then used for the diagnostic methods of the present invention.

The present invention employs definitions commonly used in the art with specific reference to the gene described in the present application.

Methods of Use: Nucleic Acid Diagnosis and Diagnostic Kits

In order to detect the presence of an BMPR2 allele that is indicative of vasoreactivity, a biological sample such as blood is prepared and analyzed for the presence or absence of the vasoreactive alleles of BMPR2. Results of these tests and interpretive information are returned to the health care provider for communication to the tested individual. Such diagnoses may be performed by diagnostic laboratories, or, alternatively, diagnostic kits are manufactured and sold to health care providers or to private individuals for self-diagnosis. Diagnostic or prognostic tests can be performed as described herein or using well known techniques, such as described in U.S. Pat. No. 5,800,998, incorporated herein by reference.

Initially, the screening method involves amplification of the relevant BMPR2 sequences. In another preferred embodiment of the invention, the screening method involves a non-PCR based strategy. Such screening methods include two-step label amplification methodologies that are well known in the art. Both PCR and non-PCR based screening strategies can detect target sequences with a high level of sensitivity.

The most popular method used today is target amplification. Here, the target nucleic acid sequence is amplified with polymerases. One particularly preferred method using polymerase-driven amplification is the polymerase chain reaction (PCR). The polymerase chain reaction and other polymerase-driven amplification assays can achieve over a million-fold increase in copy number through the use of polymerase-driven amplification cycles. Once amplified, the resulting nucleic acid can be sequenced or used as a substrate for DNA probes.

When the probes are used to detect the presence of the target sequences (for example, in screening for pulmonary arterial hypertension), the biological sample to be analyzed, such as blood or serum, may be treated, if desired, to extract the nucleic acids. The sample nucleic acid may be prepared in various ways to facilitate detection of the target sequence; e.g. denaturation, restriction digestion, electrophoresis or dot blotting. The targeted region of the analyte nucleic acid usually must be at least partially single-stranded to form hybrids with the targeting sequence of the probe. If the sequence is naturally single-stranded, denaturation will not be required. However, if the sequence is double-stranded, the sequence will probably need to be denatured. Denaturation can be carried out by various techniques known in the art.

Analyte nucleic acid and probe are incubated under conditions which promote stable hybrid formation of the target sequence in the probe with the putative targeted sequence in the analyte. The region of the probes which is used to bind to the analyte can be made completely complementary to the targeted region of human chromosome 2. Therefore, high stringency conditions are desirable in order to prevent false positives. However, conditions of high stringency are used only if the probes are complementary to regions of the chromosome which are unique in the genome. The stringency of hybridization is determined by a number of factors during hybridization and during the washing procedure, including temperature, ionic strength, base composition, probe length, and concentration of formamide. These factors are outlined in, for example, Maniatis et al., 1982 and Sambrook et al., 1989. Under certain circumstances, the formation of higher order hybrids, such as triplexes, quadraplexes, etc., may be desired to provide the means of detecting target sequences.

Detection, if any, of the resulting hybrid is usually accomplished by the use of labeled probes. Alternatively, the probe may be unlabeled, but may be detectable by specific binding with a ligand which is labeled, either directly or indirectly. Suitable labels, and methods for labeling probes and ligands are known in the art, and include, for example, radioactive labels which may be incorporated by known methods (e.g., nick translation, random priming or kinasing), biotin, fluorescent groups, chemiluminescent groups (e.g., dioxetanes, particularly triggered dioxetanes), enzymes, antibodies and the like. Variations of this basic scheme are known in the art, and include those variations that facilitate separation of the hybrids to be detected from extraneous materials and/or that amplify the signal from the labeled moiety. A number of these variations are reviewed in, e.g., Matthews and Kricka, 1988; Landegren et al., 1988; Mittlin, 1989; U.S. Pat. No. 4,868,105, and in EPO Publication No. 225,807.

As noted above, non-PCR based screening assays are also contemplated in this invention. This procedure hybridizes a nucleic acid probe (or an analog such as a methyl phosphonate backbone replacing the normal phosphodiester), to the low level DNA target. This probe may have an enzyme covalently linked to the probe, such that the covalent linkage does not interfere with the specificity of the hybridization. This enzyme-probe-conjugate-target nucleic acid complex can then be isolated away from the free probe enzyme conjugate and a substrate is added for enzyme detection. Enzymatic activity is observed as a change in color development or luminescent output resulting in a 103-106 increase in sensitivity. For an example relating to the preparation of oligodeoxynucleotide-alkaline phosphatase conjugates

Two-step label amplification methodologies are known in the art. These assays work on the principle that a small ligand (such as digoxigenin, biotin, or the like) is attached to a nucleic acid probe capable of specifically binding BMPR2 mutations. Allele specific probes are also contemplated within the scope of this example and exemplary allele specific probes include probes encompassing the predisposing or potentially predisposing mutations summarized in herein.

In one example, the small ligand attached to the nucleic acid probe is specifically recognized by an antibody-enzyme conjugate. In one embodiment of this example, digoxigenin is attached to the nucleic acid probe. Hybridization is detected by an antibody-alkaline phosphatase conjugate which turns over a chemiluminescent substrate. For methods for labeling nucleic acid probes according to this embodiment see Martin et al., 1990. In a second example, the small ligand is recognized by a second ligand-enzyme conjugate that is capable of specifically complexing to the first ligand. A well known embodiment of this example is the biotin-avidin type of interactions. For methods for labeling nucleic acid probes and their use in biotin-avidin based assays see Rigby et al., 1977 and Nguyen et al., 1992.

It is also contemplated within the scope of this invention that the nucleic acid probe assays of this invention will employ a cocktail of nucleic acid probes capable of detecting BMPR2 mutations. Thus, in one example to detect the presence of BMPR2 mutations in a cell sample, more than one probe complementary to a BMPR2 mutation is employed and in particular the number of different probes is alternatively 2, 3, or 5 different nucleic acid probe sequences. In another example, to detect the presence of mutations in the BMPR2 gene sequence in a patient, more than one probe complementary to BMPR2 is employed where the cocktail includes probes capable of binding to the allele-specific mutations identified in populations of patients with alterations in BMPR2. In this embodiment, any number of probes can be used, and will preferably include probes corresponding to the major gene mutations identified as predisposing an individual to diabetes. Some candidate probes contemplated within the scope of the invention include probes that include the allele-specific mutations identified herein and those that have the BMPR2 regions corresponding to SEQ ID NO:1 and SEQ ID NO:2 both 5′ and 3′ to the mutation site.

Methods of Use: Peptide Diagnosis and Diagnostic Kits

Vasoreactivity can also be detected on the basis of the alteration of wild-type BMPR2 polypeptide. The disclosed method is preferably carried out using a kit designed or adapted to detect one or more BMPR2 polypeptide mutations and/or one or more BMPR2 nucleic acid mutations. An example would be a kit for detecting a variety of mutated BMPR2 nucleic acids. Many such kits, and methods for using them are known. Peptide diagnostic or prognostic tests can be performed as described herein or using well known techniques, such as described in U.S. Pat. No. 5,800,998, incorporated herein by reference. For example, such alterations can be determined by sequence analysis in accordance with conventional techniques. More preferably, antibodies (polyclonal or monoclonal) are used to detect differences in, or the absence of, BMPR2 peptides. The antibodies may be prepared in accordance with conventional techniques. Other techniques for raising and purifying antibodies are well known in the art and any such techniques may be chosen to achieve the preparations claimed in this invention. In a preferred embodiment of the invention, antibodies will immunoprecipitate BMPR2 proteins or fragments of the BMPR2 protein from solution as well as react with BMPR2 peptides on Western or immunoblots of polyacrylamide gels. In another preferred embodiment, antibodies will detect BMPR2 proteins and protein fragments in paraffin or frozen tissue sections, using immunocytochemical techniques.

Preferred embodiments relating to methods for detecting BMPR2 or its mutations include enzyme linked immunosorbent assays (ELISA), radioimmunoassays (RIA), immunoradiometric assays (IRMA) and immunoenzymatic assays (IEMA), including sandwich assays using monoclonal and/or polyclonal antibodies. Exemplary sandwich assays are described by David et al. in U.S. Pat. Nos. 4,376,110 and 4,486,530, hereby incorporated by reference.

The following Examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention.

### EXAMPLES

Methods. A study was conducted utilizing a patient database developed by the Utah Pulmonary Hypertension Genetics Project, containing information relating to the genetics of pulmonary hypertension. Consecutive patients were sought at the Pulmonary Hypertension Center at LDS Hospital and at biannual meetings of the Pulmonary Hypertension Association. Patients provided written informed consent according to a protocol approved by the Institutional Review Board of the LDS Hospital. Blood samples were obtained and DNA was extracted from lymphocytes via salting-out protocol (PureGene; Gentra Systems, Minneapolis, Minn.). Complete medical records were obtained to confirm the diagnosis of idiopathic or familial pulmonary arterial hypertension according to consensus standards [McGoon, 2004]. In brief the diagnosis of idiopathic pulmonary arterial hypertension required that a consultant with expertise in pulmonary vascular disease confirm the following: (1) mean pulmonary artery pressure >25 mmHg with a pulmonary capillary wedge pressure ≦15 mmHg, both measured at rest by right heart catheterization and (2) the exclusion of other disorders known to cause pulmonary hypertension by objective tests, e.g. ventilation and perfusion lung scans to exclude pulmonary embolism, contrast echocardiography and measurements of oxygen saturation during cardiac catheterization to exclude intra-cardiac shunting and echocardiography and cardiac catheterization to exclude left heart disease. The diagnosis of familial pulmonary arterial hypertension required that a consultant with expertise confirm the diagnosis of pulmonary arterial hypertension in two or more members of the same family.

Patient Cohort. The patient data-base was queried and a cohort of patients who had the diagnosis of idiopathic pulmonary arterial hypertension or familial pulmonary arterial hypertension (n=164) was identified. Patients were excluded if (1) there was no DNA available for analysis (n=45) or (2) complete data from a test of vasoreactivity was not available (n=48). The study cohort (n=71) represented patients who were evaluated at 21 medical centers.

Vasoreactivity testing. Vasoreactivity tests were performed according to each individual hospital protocol and choice of vasodilator. The dose of epoprostenol, adenosine, nifedipine, nitroprusside, prostaglandin E2 or phentolamine was increased until either an intolerable side effect occurred or vasoreactivity was observed [Groves B, 1993]. Nitric oxide was inhaled and the hemodynamic response was measured shortly thereafter according to local institutional protocol [Sitbon, 1995; Sitbon, 1998]. The first test of vasoreactivity was used for each patient. Vasoreactivity was defined by recent consensus guidelines as a decrease in mean pulmonary artery pressure of at least 10 mmHg to a level less than or equal to 40 mmHg with no change or an increased cardiac output [Badesch, 2004; Galie, 2004].

Molecular analysis. Genomic DNA was screened for mutations in BMPR2 by PCR amplification of exons and analysis of amplicons using dye binding high-resolution thermal denaturation as described previously [McKinney JT 2004; Reed GH 2004; Havlena, 2004]. Specific mutations were confirmed by sequencing the BMPR2 complementary DNA (c DNA) using big dye terminator chemistry and 17 pairs of overlapping primers. Parts of exon 12 were not sequenced because no deviations from wild type melting profiles were identified by dye binding high-resolution thermal denaturation.

Statistical analysis. Data were analyzed using Statistica (Tulsa, Okla.) for means and distribution attributes. The Fisher's exact test was used to compare categorical variables among the cohorts, which were grouped according to mutational status (non-synonymous mutation or wild type) and p<0.01 was chosen to account for multiple analyses. Comparisons of vasoreactivity between patients with and without gene mutations were performed using the Fisher's exact test. Tests were considered significant if p values were <0.05.

Clinical Data. The study population included 52 patients with sporadic idiopathic pulmonary arterial hypertension and 19 with familial pulmonary arterial hypertension (Table 1, below).

TABLE 1
Characteristics of the study participants
at the time of vasoreactivity testing

| | | | No BMPR2 |
|---|---|---|---|
| | | Non- | mutation |
| | | synonymous | or synonymous |
| | | BMPR2 | BMPR2 |
| | All | mutations | mutation |
| | (n = 71) | (n = 24) | (n = 47) |
| Age yrs. x̅ ± SD | 37.4 ± 11.6 | 34.8 ± 12.1 | 38.8 ± 11.1 |
| Sex: M/F | 14/57 | 8/16 | 6/41 |
| Height (cm), | 166.4 ± 9.5 | 165.7 ± 10.8 | 166.7 ± 8.9 |
| x̅ ± SD | | | |
| Weight (kg), | 76.0 ± 17.4 | 78.4 ± 17.2 | 74.7 ± 17.5 |
| x̅ ± SD | | | |
| Race or ethnic | | | |
| group, n (%) | | | |
| White | 66 (93) | 23 (96) | 43 (91) |
| Black | 1 (1.5) | 0 (0) | 1 (2) |
| Asian | 3 (4.5) | 1 (4) | 2 (4) |
| Hispanic | 1 (1.5) | 0 (0) | 1 (2) |
| Family history, n (%) | 19 (27) | 13 (54) | 6 (13)† |
| Baseline* x̅ ± SD | | | |
| MPAP mmHg | 58.6 ± 11.2 | 60.3 ± 11.4 | 57.8 ± 11.1 |
| MRAP mmHg | 10.2 ± 6.5 | 10.6 ± 5.8 | 10.0 ± 6.9 |
| PAWP mmHg | 9.2 ± 3.4 | 9.0 ± 3.4 | 9.4 ± 3.6 |
| PVR Wood units | 13.9 ± 5.9 | 15.2 ± 5.5 | 12.9 ± 6.1 |
| CO L. min⁻¹ | 4.0 ± 1.2 | 3.8 ± 1.3 | 4.1 ± 1.2 |
| CI L. min^{-1.}m⁻² | 2.2 ± 0.7 | 2.0 ± 0.7 | 2.3 ± 0.7 |
| NYHA functional | | | |
| class | | | |
| II n (%) | 1 (1.5) | 0 (0) | 1 (2) |
| III n (%) | 69 (97) | 23 (96) | 46 (98) |
| IV n (%) | 1 (1.5) | 1 (4) | 0 (0) |

*MPAP—mean pulmonary artery pressure; MRAP—mean right atrial pressure; PAWP pulmonary artery wedge pressure; PVR—pulmonary vascular resistance; CO—cardiac output; CI—cardiac index
†p = .0088 by 2-tailed Fisher's exact test for the comparison of family history for patients with nonsynonymous BMPR2 mutations and patients without a BMPR2 mutation or with synonymous BMPR2 mutations.

Fifty-seven of 71 were women. The mean (±SD) age of the 71 subjects was 37±11 years (range 14-64 years). Sixty-nine of 71 patients were New York Heart Association functional class III at the time of the first vasoreactivity test. Patients with non-synonymous BMPR2 mutations (n=24) did not differ from those without a BMPR2 mutation or with a synonymous BMPR2 mutation with respect to age, sex, height, weight, race, baseline hemodynamics, or New York Heart Association functional class at the time of vasoreactivity testing. Patients with non-synonymous BMPR2 mutations were more likely to have familial pulmonary arterial hypertension than those without nonsynonymous BMPR2 mutations (13 of 24 vs. 6 of 47, p=0.0088).

Epoprostenol, adenosine, nitric oxide, and high doses of nifedipine were used for 94 percent of the vasoreactivity tests (Table 2).

TABLE 2
Vasodilators and dose characteristics used for vasoreactivity tests

| | | |
|---|---|---|
| Vasodilator | n (%) | Median Maximal Dose (range) |

| | | |
|---|---|---|
| Epoprostenol | 23 (32) | 6.5 ng/kg/min (2-16) |
| Adenosine | 19 (27) | 200 μg/kg/min (100-350) |
| Nitric oxide | 15 (21) | 40 ppm (20-80) |
| Nifedipine | 10 (14) | 90 mg ** (40-240) |
| Other * | 4 (6) | — |

* Other vasodilators included nitroprusside 1.4 mcg/kg/min and 2 mcg/kg/min (n = 2), prostaglandin E2 175 mcg/kg/min (n = 1), and phentolamine 5 mg (n = 1)
** Cumulative dose

The median maximal dose of epoprostenol was 6.5 ng/kg/min (range 2-16 ng/kg/min); the median maximal dose of adenosine was 200 μg/kg/min (range 100-350 μg/kg/min); and the median maximal dose of nifedipine was 90 mg (range 40-240 mg). Nitric oxide doses ranged between 20 and 80 ppm.

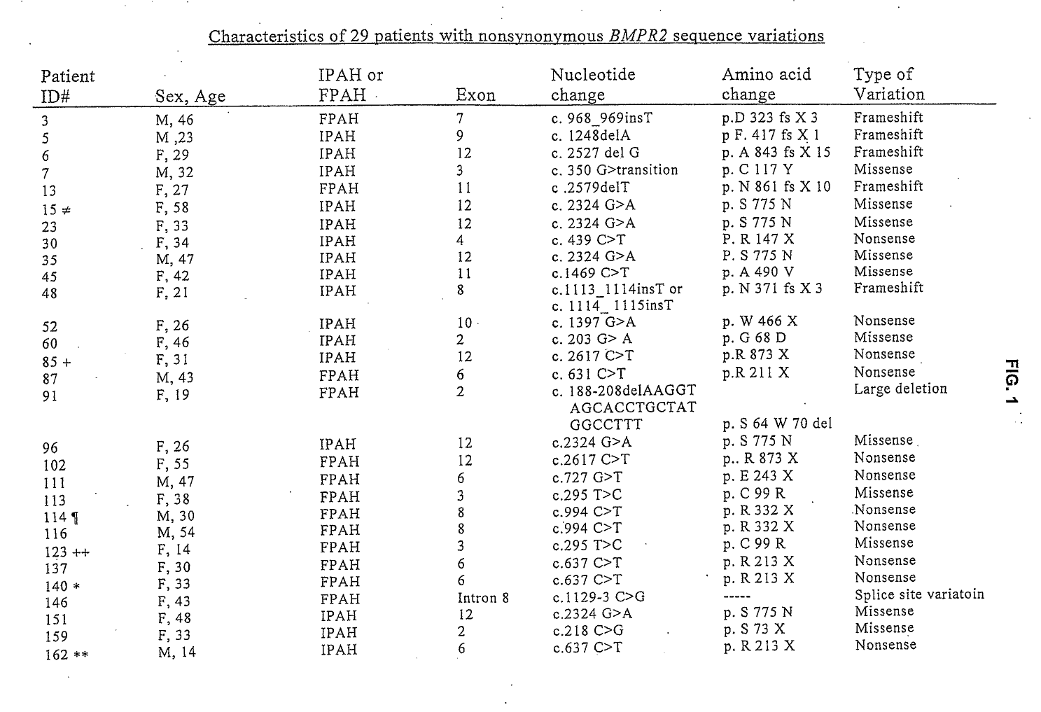

Molecular data. Mutations were identified in 40 of 71 patients with idiopathic (n=27 of 52) or familial (n=13 of 19) pulmonary arterial hypertension. Twenty-four of 40 patients had non-synonymous BMPR2 mutations predicted to alter the coding sequence of BMPR2 and the protein product (FIG. 1). These 24 mutations included eleven nonsense, six missense, five frameshift, one large deletion, and one mutation at the intron boundary before exon 9. One pair of sisters and one apparently unrelated man shared the same mutation; and one mother and her daughter shared the same mutation. Two apparently unrelated individuals shared the same mutation. The remaining 17 subjects had novel BMPR2 mutations predicted to alter the protein product. Sixteen patients, all with idiopathic pulmonary arterial hypertension and none with familial pulmonary arterial hypertension, had common polymorphisms not predicted to alter the amino acid sequence of the BMPR type-2 receptor (Table 3) [Morisaki 2004; Machado, 2001; Trembath personal communication].

TABLE 3
Common synonymous BMPR2 polymorphisms
identified in sixteen patients

| | | | Amino acid | |
|---|---|---|---|---|
| n | Location | Nucleotide change | change | Frequency |
| 4 | Exon 5 | c. 600 A > C | p. L 200 L | NK † |
| 4 | Exon 12 | c. 2324 G > A | p. S 775 N | 5% † |
| 7 | Exon 12 | c. 2811 G > A | p. R 937 R | 14% * |
| 1 | Exon 12 | c. 2811 G > A and | p. S 775 N and | |
| | | c. 2324 G > A | p. R 937 R | |

† Trembath R. Personal communication, NK = not known
* Morisaki, et al, 2004 and Machado, et al, 2001

Vasoreactivity data. Overall, 15 of 71 patients (21%) were vasoreactive. Vasoreactivity occurred more commonly among patients without a BMPR2 mutation. None of the 24 patients with non-synonymous BMPR2 mutations demonstrated vasoreactivity whereas 15 of 47 (32 percent) with either BMPR2 synonymous mutations or no mutation demonstrated acute vasoreactivity (p=0.001). This finding was not changed by excluding non-synonymous mutations (n=16) from the analysis (11 of 31 without BMPR2 mutations versus 0 of 24 with non-synonymous BMPR2 mutations demonstrated vasoreactivity, p=0.001). Only 4 of 40 (10 percent) patients with either synonymous or non-synonymous BMPR2 mutations demonstrated acute vasoreactivity whereas eleven of 31 patients (35 percent) without BMPR2 mutations demonstrated acute vasoreactivity (p=0.010). It is possible that individuals with familial pulmonary arterial hypertension had undetected BMPR2 mutations. Assuming that there were undetected mutations in the six familial patients for whom we did not find BMPR2 mutations did not change the finding that vasoreactivity was unlikely. In this scenario only 1 of 30 individuals with either non-synonymous BMPR2 mutation (n=24) or familial pulmonary arterial hypertension without a detectable BMRP2 mutation (n=6) demonstrated vasoreactivity; whereas 14 of 41 individuals with either wild type BMPR2 (n=31) or synonymous BMPR2 mutations (n=10) demonstrated vasoreactivity (p=0.002).

Conclusions. The above results suggest that BMPR2 mutations are predictive of non-vasoreactivity. The patient population was drawn from a large population of patients with typical features of idiopathic and familial pulmonary arterial hypertension. Most were middle-aged women with symptoms indicative of New York Heart Association Functional Class III disease. The patients all had severe pulmonary arterial hypertension at the time of their initial vasoreactivity test. Furthermore the majority of patients were identified and studied at major referral centers with experience in the diagnosis and evaluation of pulmonary arterial hypertension. Physicians used drugs commonly accepted for tests of acute vasoreactivity [Badesch, 2004], and the protocols for vasoreactivity tests employed similar endpoints. Furthermore vasoreactivity tests were performed with vasodilators titrated to doses proven to identify pulmonary arterial hypertension patients with marked vasoreactivity [Groves, 1993; Sitbon, 1995; Sitbon, 1998; Ricciardi, 2001; Weir, 1989].

In summary, patients with severe idiopathic or familial pulmonary arterial hypertension and non-synonymous BMPR2 mutations are unlikely to display vasoreactivity, suggesting that vasoreactivity tests may be unnecessary for such patients, and that the costs and attendant risks of vasoreactivity tests may be avoided.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

### REFERENCES

Badesch D B, Abman S H, Ahearn G S, et al. Medical Therapy for Pulmonary Arterial Hypertension. ACCP evidence-based clinical practice guidelines. CHEST 2004; 126:35S-62S.
Cogan, J D, Vnencak-Jones C L, Phillips J A, et al. Gross BMPR2 gene rearrangements constitute a new cause for primary pulmonary hypertension. Genet Med 2005; 7:168-174.
Galie N, Seeger W, Naeije R, et al. Comparative analysis of clinical trials and evidence-based treatment algorithm in pulmonary arterial hypertension. J Am Coll Cardiol 2004; 43:81S-88S.
Groves B M, Badesch D B, Turkevich D, et al. Correlation of acute prostacyclin response in primary (unexplained) pulmonary hypertension with efficacy of treatment with calcium channel blockers and survival. In: Weir K, ed. Ion flux in pulmonary vascular control. New York, N.Y.: Plenum Press, 1993; 317-330.
Havlena G T, Elliott C G, Carlquist J. Comprehensive mutation scanning of the BMPR2 gene using high resolution melting curve analysis. Am J Respir Crit Care Proceedings 2004; 169:A397.
Koehler R, Grunig E, Pauciulo M W, et al. Low frequency of BMPR2 mutations in a German cohort of patients with sporadic idiopathic pulmonary arterial hypertension. J Med Genet. 2004; 41:e127.
Lane K B, Machado R D, Pauciulo M W, et al. Heterozygous germline mutations in BMPR2, encoding a TGF-beta receptor, cause familial primary pulmonary hypertension. The international PPH consortium. Nat Genet. 2000; 26:81-4.
Lee S D, Shrayer K R, Markham N E, Cool C D, Voelkel N F, Tuder R M. Monoclonal endothelial cell proliferation is present in primary but not secondary pulmonary hypertension. J Clin Invest 1998; 101:927-934.
Loscalzo J. Genetic clues to the cause of primary pulmonary hypertension. N Engl J Med 2001; 345:367-371.
Loyd J E, Atkinson J B, Pietra G G, Virmani R, Newman J H. Heterogeneity of pathologic lesions in familial primary pulmonary hypertension. Am Rev Respir Dis 1988; 138:952-7.
Machado R D, Pauciulo M W, Thomsen J R, et al. BMPR2 haploinsufficiency as the inherited molecular mechanism for primary pulmonary hypertension. Am J Hum Genet 2001; 68:92-102.
McCaffrey T A, Consigli S, Du B, et al. Decreased type II/type I TGF-beta receptor ratio in cells derived from human atherosclerotic lesions. Conversion from an antiproliferative to profibrotic response to TGF-beta1. J Clin Invest 1995; 96:2667-2675.
McGoon M, Gutterman D, Steen V, et al. Screening, early detection, and diagnosis of pulmonary arterial hypertension. CHEST 2004; 126:14S-34S.
McKinney J T, Longo N, Hahn S H, Matern D, Rinaldo P, Strauss Aw, Dobrowolski S F. Rapid, comprehensive screening of the human medium chain acyl-CoA dehydrogenase gene. Mol Genet Metab 2004; 82:112-120.
Morisaki H, Nakanishi N, Kyotani S, et al. BMPR2 mutations found in Japanese patients with familial and sporadic primary pulmonary hypertension. Hum Mutat 2004; 23:632.
Newman J H, Trembath R C, Morse J A, et al. Genetic basis of pulmonary arterial hypertension. J Am Coll Cardiol 2004; 43:33S-39S.
Ogata M, Ohe M, Shirato K, et al. Effects of a combination therapy of anticoagulant and vasodilator on the long-term prognosis of primary pulmonary hypertension. Jpn Circ J 1993; 57:63-69.
Optiz C F, Wensel R, Bettmann M, et al. Assessment of the vasodilator response in primary pulmonary hypertension: comparing prostacyclin and iloprost administered by either infusion or inhalation. Eur Heart J 2003; 24:356-65.
Raffy O, Azarian R, Brenot F, et al. Clinical significance of the pulmonary vasodilator response during short term infusion of prostacyclin in primary pulmonary hypertension. Circulation 1996; 93:484-488.
Reed G H, Wittwer C T. Sensitivity and specificity of single-nucleotide polymorphism scanning by high-resolution melting analysis. Clin Chem 2004; 50:1748-54.
Ricciardi M J, Knight B P, Martinez F J, et al. Inhaled nitric oxide in primary pulmonary hypertension: a safe and effective agent for predicting response to nifedipine. J Am Coll Cardiol 2001; 38:1267-68.
Rich S, Kaufmann E, Levy P S. The effect of high doses of calcium-channel blockers on survival in primary pulmonary hypertension. N Engl J Med 1992; 327:76-81.
Rubin L J. Primary pulmonary hypertension. N Engl J Med 1997; 336:111-17.
Rubin L J. Introduction: diagnosis and management of pulmonary arterial hypertension: ACCP evidence-based clinical practice guidelines. Chest 2004; 126:7S-10S.
Runo J R, Loyd J E. Primary pulmonary hypertension. Lancet 2003; 361:1533-1544.
Schrader B J, Inbar S, Kaufmann L, et al. Comparison of the effects of adenosine and nifedipine in pulmonary hypertension. J Am Coll Cardiol 1992; 19:1060-64.
Sitbon O, Humbert M, Jagot J L, et al. Inhaled nitric oxide as a screening agent for safely identifying responders to oral calcium-channel blockers in primary pulmonary hypertension. Eur Respir J 1998; 12:265-70.
Sitbon O, Brenot F, Denjean A, et al. Inhaled nitric oxide as a screening vasodilator agent in primary pulmonary hypertension: a dose-response study and comparison with prostacyclin. Am J Respir Crit Care Med 1995; 151:384-89.
Thomson J R, Machado R D, Pauciulo M W, et al. Sporadic primary pulmonary hypertension is associated with germline mutations of the gene encoding BMPR-II, a receptor member of the TGF-β family. J. Med Genet 2000; 37:741-745.
Weir E K, Rubin L J, Ayres S M, et al. The acute administration of vasodilators in primary pulmonary hypertension. Am Rev Respir Dis 1989; 140:1623-1630.
West J, Fagan K, Steudel S W, et al. Pulmonary hypertension in transgenic mice expressing a dominant-negative BMPR II gene in smooth muscle. Circ Res. 2004; 94:1109-1114.
Wood P. Pulmonary hypertension with special reference to the vasoconstrictive factor. Br Heart J 1958; 20:557-70.

1. A method of determining the vasoreactivity of a subject, comprising:
obtaining from a subject a sample comprising a nucleic acid sequence of the BMPR2 gene or amino acid sequence of the BMPR2 gene;
determining the presence or absence of a non-synonymous mutation in the BMPR2 nucleic acid or amino acid sequence, and
correlating the presence of a non-synonymous mutation with non-vasoreactivity or the absence of a non-synonymous mutation with vasoreactivity.

2. The method of claim 1, wherein the subject has been diagnosed with pulmonary arterial hypertension.

3. The method of claim 1, wherein the non-synonymous mutation in the BMPR2 nucleic acid or amino acid sequence corresponds to a mutation at any one or more of the following nucleotide positions of SEQ ID NO:1: 218, 354, 355, 367, 439, 504, 689, 958, 994, 1042, 1076, 1129, 1191, 1258, 1454, 1535, 1557, 1749, 2292, 2408, 2579, and 2695.

4. The method of claim 1, wherein the non-synonymous mutation in the BMPR2 nucleic acid sequence or amino acid sequence corresponds to a mutation characterized as any one or more of the following, or a complement thereof: C218G, T354G, T367C, T367A, C439T, C994T, G1042A, T1258C, A1454G, A1535C, T1557A, C2695T.

5. The method of claim 1, wherein the non-synonymous BMPR2 mutation in the BMPR2 nucleic acid sequence or amino acid sequence corresponds to a mutation at any one or more of the following amino acid positions of SEQ ID NO:2: 73, 118, 123, 143, 332, 348, 420, 485, 512, 519, and 899.

6. The method of claim 1, wherein the non-synonymous mutation in the BMPR2 nucleic acid sequence or amino acid sequence corresponds to a mutation characterized as any one or more of the following mutations in the BMPR2 protein: 73term, 118W, 123R, 123S, 143term, 332term, 3481, 420R, 485A, 512GQterm, 519K, 899term.

7. A method of treating a patient diagnosed with pulmonary arterial hypertension, comprising:
obtaining from a subject a sample comprising a nucleic acid sequence of the BMPR2 gene or amino acid sequence of the BMPR2 gene;
determining the presence or absence of a non-synonymous mutation in the BMPR2 nucleic acid or amino acid sequence;
correlating the presence of a non-synonymous mutation with non-vasoreactivity status of the patient or the absence of a non-synonymous mutation with vasoreactivity status of the patient; and
providing a clinical recommendation to the patient based upon the non-vasoreactivity or vasoreactivity status of the patient.

8. The method of claim 7, wherein the non-synonymous mutation in the BMPR2 nucleic acid or amino acid sequence corresponds to a mutation at any one or more of the following nucleotide positions of SEQ ID NO:1: 218, 354, 355, 367, 439, 504, 689, 958, 994, 1042, 1076, 1129, 1191, 1258, 1454, 1535, 1557, 1749, 2292, 2408, 2579, and 2695.

9. The method of claim 7, wherein the non-synonymous mutation in the BMPR2 nucleic acid sequence or amino acid sequence corresponds to a mutation characterized as any one or more of the following, or a complement thereof: C218G, T354G, T367C, T367A, C439T, C994T, G1042A, T1258C, A1454G, A1535C, T1557A, C2695T.

10. The method of claim 7, wherein the non-synonymous BMPR2 mutation in the BMPR2 nucleic acid sequence or amino acid sequence corresponds to a mutation at any one or more of the following amino acid positions of SEQ ID NO:2: 73, 118, 123, 143, 332, 348, 420, 485, 512, 519, and 899.

11. The method of claim 7, wherein the non-synonymous mutation in the BMPR2 nucleic acid sequence or amino acid sequence corresponds to a mutation characterized as any one or more of the following: 73term, 118W, 123R, 123S, 143term, 332term, 3481, 420R, 485A, 512GQterm, 519K, 899term.

12. An isolated polynucleotide comprising a sequence of nucleic acids containing a polymorphism selected from the group consisting of: 188-208del121, G203A, T295C, A600C, 968_{—}969insT, 1113_{—}1114insT, C1469T, and 2527delG.

13. An antibody having specificity to a protein encoded by any one of the mutations of claim 12.

14. A kit for determining the vasoreactivity of a subject, comprising reagents for detecting the presence or absence of a non-synonymous mutation in the BMPR2 nucleic acid or BMPR2 protein of the subject.
